(19) United States
(12) Patent Application Publication
(10) Pub. No.: US 2026/0249069 A1
(43) Pub. Date: Aug. 27, 2026

(54) CIRCULATION SUPPORT DEVICES, SYSTEMS, AND METHODS

(71) Applicant: Boston Scientific Scimed, Inc., Maple Grove, MN (US)

(72) Inventors: Gavin O'Brien, Sutton (IE); Tim O'Connor, Galway (IE); Chris Cullen, Galway (IE); Richard O'Sullivan, Turloughmore (IE)

(73) Assignee: Boston Scientific Scimed, Inc., Maple Grove, MN (US)

(21) Appl. No.: 19/546,920

(22) Filed: Feb. 23, 2026

Related U.S. Application Data
(60) Provisional application No. 63/762,360, filed on Feb. 24, 2025.

(51) Int. Cl. A61M 60/174; A61M 60/13; A61M 60/216; A61M 60/865; A61M 60/871

(52) U.S. Cl. CPC A61M 60/174; A61M 60/13; A61M 60/216; A61M 60/865; A61M 60/871

(57) ABSTRACT

A mechanical circulatory support system may include a blood pump and an elongate shaft coupled to the blood pump. The blood pump may be configured to extend into a ventricle of a heart of a patient. The elongate shaft may be configured to be pulled through an aorta of the patient and into an axillary artery of the patient while the blood pump is in the heart of the patient. The blood pump may be a large-bore blood pump. The elongate shaft may be detachable from the blood pump. The elongate shaft extending through the axillary artery of the patient may be coupled with a controller configured to operate the blood pump while the patient is ambulatory.

### CROSS REFERENCE TO RELATED APPLICATIONS

This application claims the benefit of U.S. Provisional Patent Application Serial No. 63/762,360 filed on February 24, 2025, the disclosure of which is incorporated herein by reference.

### TECHNICAL FIELD

The present disclosure pertains to cardiac devices and systems. More specifically, the present disclosure relates to devices, systems, and methods of and/or for use of cardiac devices and systems.

### BACKGROUND

A wide variety of medical devices have been developed for medical use including, for example, medical devices utilized to provide transient support in patients with compromised heart function or cardiac output. Generally, such devices, which may be referred to as percutaneous circulatory support devices or percutaneous blood pumps, are delivered to a patient's heart through the vasculature of the patient via a femoral access site. However, there is an ongoing need to provide alternative configurations of vascular access devices for use with percutaneous blood pumps, and methods of gaining vascular access and placement of the percutaneous blood pump into a patient’s vasculature.

### BRIEF SUMMARY

This disclosure provides design, material, manufacturing method, and use alternatives for medical devices, including cardiac devices and devices used therewith.

A first example may include a mechanical circulatory support system comprising a blood pump configured to extend into a ventricle of a heart of a patient and an elongate shaft coupled to the blood pump and configured to be pulled through an aorta of the patient and into an axillary artery of the patient while the blood pump is in the heart of the patient, wherein the blood pump is a large-bore blood pump.

Alternatively or additionally to any of the examples above, the blood pump may have an outer diameter of at least 8 millimeters (mm) and may be configured to achieve a flow rate of at least 5.5 liters per minute (L/min).

Alternatively or additionally to any of the examples above, the elongate shaft may be detachable from the blood pump and is configured to be removed from the patient through the axillary artery.

Alternatively or additionally to any of the examples above, the elongate shaft may have a length sufficient to extend from the blood pump in the ventricle of the heart of the patient to an access site through the axillary artery.

Alternatively or additionally to any of the examples above, the elongate shaft may have a length that is shorter than a distance from a femoral artery access site, through the aorta, and to a location in the aorta proximate the heart of the patient.

Alternatively or additionally to any of the examples above, the system may include a power source configured to releasably couple with the elongate shaft.

Alternatively or additionally to any of the examples above, the system may include a controller configured to couple with the elongate shaft and control operation of the blood pump, wherein the controller is configured to be secured to a body of the patient.

Alternatively or additionally to any of the examples above, the system may include a retrieval snare configured to access the elongate shaft in the aorta of the patient and move the elongate shaft through the axillary artery.

Alternatively or additionally to any of the examples above, the system may include a delivery sheath configured to receive the blood pump and the elongate shaft, wherein the delivery sheath is configured to extend from a femoral artery access site, through the aorta, and to a location in the aorta proximate the heart of the patient.

Alternatively or additionally to any of the examples above, the system may include a retrieval sheath configured to access the elongate shaft extending through the axillary artery and move the elongate shaft into the aorta, wherein the retrieval sheath is configured to extend over the blood pump during removal of the blood pump from the patient through a femoral artery access site.

In an example, a method may include inserting a pump to a heart of a patient through a femoral artery access site using a delivery sheath, removing the delivery sheath from an

elongate shaft extending from the pump while leaving the pump in place, accessing the elongate shaft of the pump via an axillary artery of the patient, and coupling the elongate shaft with a controller.

Alternatively or additionally to any of the examples above, the method may include positioning the elongate shaft through an axillary artery access site.

Alternatively or additionally to any of the examples above, accessing the elongate shaft of the pump via the axillary artery may comprise snaring the elongate shaft while the elongate shaft is extending along an aorta of the patient.

Alternatively or additionally to any of the examples above, the method may include pulling the elongate shaft through the axillary artery.

Alternatively or additionally to any of the examples above, the method may include initiating operation of the pump to treat a cardiogenic shock condition of the patient while allowing patient to be ambulatory.

Alternatively or additionally to any of the examples above, the method may include detaching the elongate shaft from the pump and removing the elongate shaft from vasculature of the patient through the axillary artery.

Alternatively or additionally to any of the examples above, the method may include engaging the elongate shaft with a retrieval sheath extending through a femoral artery access site while the elongate shaft is extending along the axillary artery.

In another example, a method may include operating a large-bore blood pump extending into a heart of a patient via an elongate shaft coupled with the large-bore blood pump and extending through an axillary artery of the patient and removing the large-bore blood pump from the patient via a femoral artery access site.

Alternatively or additionally to any of the examples above, the method may include grasping the elongate shaft extending through the axillary artery of the patient, wherein the elongate shaft is removed from the patient via the femoral artery access site with the large-bore blood pump.

Alternatively or additionally to any of the examples above, the method may include detaching the elongate shaft from the large-bore blood pump and removing the elongate shaft from vasculature of the patient via an axillary artery access site.

The above summary of some configurations is not intended to describe each disclosed configuration or every implementation of the present disclosure. The Figures, and Detailed Description, which follow, more particularly illustrate some of these configurations.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative circulatory support system;

FIG. 2 is a schematic partial cross-section view of anatomy and a schematic side view of an illustrative mechanical circulatory support (MCS) system within the anatomy;

FIG. 3 is a schematic cross-section view of a portion of an introducer sheath within a blood vessel and a portion of an illustrative MCS system in the introducer sheath and the blood vessel;

FIG. 4 is a schematic box diagram of an illustrative method of using an MCS system;

FIG. 5 is a schematic box diagram of an illustrative method of using an MCS system;

FIGS. 6A-6E schematically depict views illustrating a portion of an illustrative technique of using an MCS system; and

FIGS. 7A-7D schematically depict views illustrating a portion of an illustrative technique of using an MCS system.

While the disclosure is amenable to various modifications and alternative forms, specifics thereof have been shown by way of example in the drawings and will be described in detail. The intention is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative configurations and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

A variety of circulatory assist or support devices are known for assisting or replacing a pumping function of a heart in a patient with severe heart failure and/or other cardiac conditions. Circulatory support devices may be configured to treat patients with cardiogenic shock, myocardial infarction, acutely decompensated heart failure, and/or other heart related conditions. Additionally or alternatively circulatory support systems or devices may support a patient during percutaneous coronary interventions and/or other procedures.

Example cardiac circulatory support systems or devices include, but are not limited to, ventricular assist devices (VADs), total artificial hearts, intra-aortic balloon pumps (IABP), and extracorporeal membrane oxygenation (ECMO) devices. Example VADs include left ventricular assist devices (LVADs), right ventricular assist devices (RVADs), and biventricular assist devices (BiVADs). A further illustrative VAD is a percutaneous ventricular assist device (PVAD), which may be inserted into a ventricle (e.g., a left ventricle or a right ventricle) of a heart of a patient via delivery through an artery (e.g., femoral, axillary, etc.) or vein and/or other suitable vasculature to the ventricle. A PVAD may be placed at a desired location of anatomy of a patient via percutaneous access and delivery, which may enable the PVAD to be used in emergency medicine, a cath lab, and/or other surgical and/or non-surgical settings.

In some examples, circulatory support systems may include a distal end having a flexible cannula that is configured to be inserted into a heart of a patient. A housing (e.g., a rigid housing) that may house one or more pumping components may extend in a proximal direction from a proximal end of the flexible cannula. One or more bends in the flexible cannula may direct the distal end of the flexible cannula through the aortic valve of the patient and into the left ventricle of the heart.

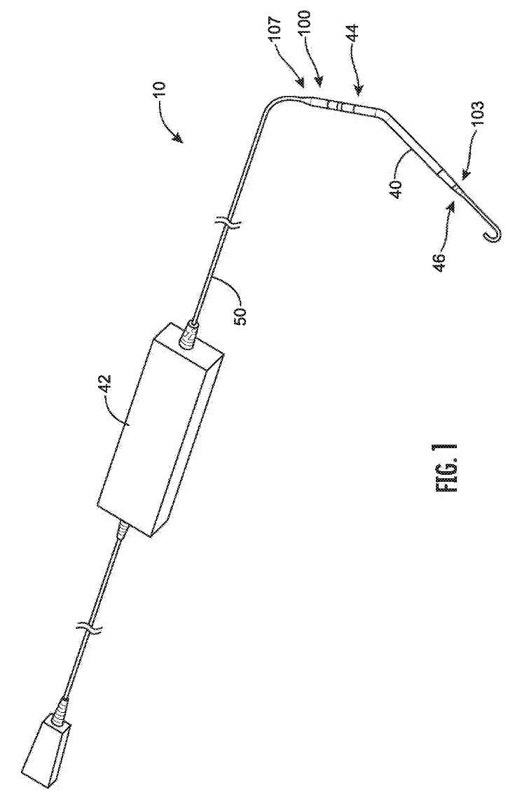

FIG. 1 depicts a schematic view of an illustrative circulatory support system 10 (e.g., a mechanical circulatory support (MCS) system). The system 10 may include a percutaneous support device (e.g., a PVAD, such as a blood pump 100 having a proximal end 107 and a distal end 103), a cannula 40 having a proximal end 44 and a distal end 46, an elongate shaft 50, and a delivery or guide sheath (not shown). In some examples, the system 10 may include a guidewire, but this is not required. The delivery or guide sheath, when included, may facilitate percutaneous delivery of the blood pump 100 and the cannula 40 to a target location or site within a patient (e.g., a target site within a heart of a patient). When positioned at a target site in a heart of a patient, the blood pump 100 may be configured to pump blood from a ventricle of the heart to vasculature of the patient to provide cardiac assistance.

The cannula 40 may be a component of the blood pump 100 and/or a separate component coupled with the blood pump 100. In some examples, although the cannula 40 may be discussed as extending from the blood pump 100 with the proximal end 44 of the cannula 40 at or proximate the distal end of the blood pump 100, the cannula 40 may be part of the blood pump 100 (e.g., formed from a component of the blood pump 100, such as an impeller housing and/or other suitable component) with the distal end 46 of the cannula 40 at the distal end 103 of the blood pump 100, but other suitable configurations are contemplated.

The system 10 may further include a proximal housing 42 coupled with (e.g., connected to) the elongate shaft 50 (e.g., a catheter, a power cable, and/or other suitable elongate tube), where the elongate shaft 50 may be coupled with the blood pump 100 (e.g., a distal end of the elongate shaft 50 may be coupled with the proximal end 107 of the blood pump 100) and one or more wires and/or shafts coupled with the blood pump 100 may extend proximally from the blood pump 100 through the elongate shaft 50. In some examples, at least the proximal housing 42, the elongate shaft 50, and/or other components of the system 10 may be configured to facilitate delivering the blood pump 100 and the cannula 40 to the target location or site within the patient. The elongate shaft 50 and the proximal housing 42 may be configured to be positioned at least partially exterior of the patient when the blood pump 100 and the cannula 40 are at the target location and the blood pump 100 is in operation. In some examples, the guidewire, when included, may also be used to facilitate the delivery of the blood pump 100 and/or the cannula 40.

The housing 42 may be any suitable type of housing. For example, the housing 42 may be or may be configured to house a power source, a portable power source, a non-portable power source, a controller, a portable controller, a non-portable controller, a wired or wireless communication module, a user interface, one or more control buttons or components, a touch screen, a display, circuitry, one or more processors, memory, a handle, and/or other suitable components configured to facilitate the use or positioning of the blood pump 100. In one example, the housing 42 may be or may include a portable power source and a portable controller.

The elongate shaft 50 may be any suitable type of tube and may have any suitable length. For example, the elongate shaft 50 may be or may house a cable that houses one or more wires (e.g., electrical wires, optical wires, drive wires, etc.), a power cable, a control cable, a wire, and/or other suitable components configured to facilitate the use or positioning of the blood pump 100. In one example, the elongate shaft 50 may be or may include one or more power cables or wires and one or more control cables or wires.

Although not shown, the system 10 may additionally or alternatively include one or both of a starter tube and a starter tube flushing line. The starter tube, when included, may couple with a cannula delivery tool (e.g., a delivery sheath) which may be configured to receive the cannula 40 prior to and/or during delivery of the blood pump 100 and the cannula 40 to a target site.

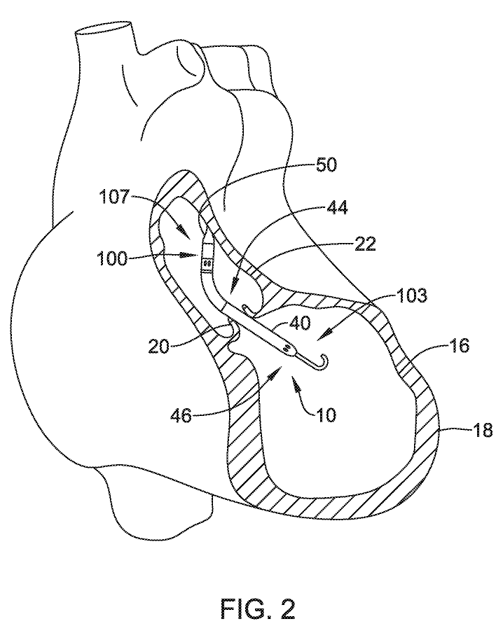

FIG. 2 depicts a schematic view of an illustrative positioning of the blood pump 100 (e.g., a percutaneous circulatory support device, such as a PVAD, etc.) in anatomy of a patient (e.g., in a left ventricle 16 in a heart 18). As depicted in FIG. 2, the blood pump 100 may be positioned with the distal end 103 thereof (e.g., when including or combined with the cannula 40) located in the left ventricle 16 of the heart 18 and the proximal end 107 of the blood pump 100 in an aorta 22, such that the blood pump 100 and the cannula 40 in combination extend across an aortic valve 20 between the left ventricle 16 and the aorta 22. With the blood pump 100 extending from the left ventricle 16 to the aorta 22 of the patient, the blood pump 100 may be configured to pump blood from the left ventricle 16 into the aorta 22 (e.g., into the ascending aorta) to assist or support blood flow circulation. Other suitable positions of the blood pump 100 relative to the anatomy of the patient are contemplated and include, but are not limited to, the distal end 103 of the blood pump 100 being positioned in or in fluid communication with a right ventricle of the heart 18 with the proximal end 107 of the blood pump 100 being positioned in a pulmonary artery.

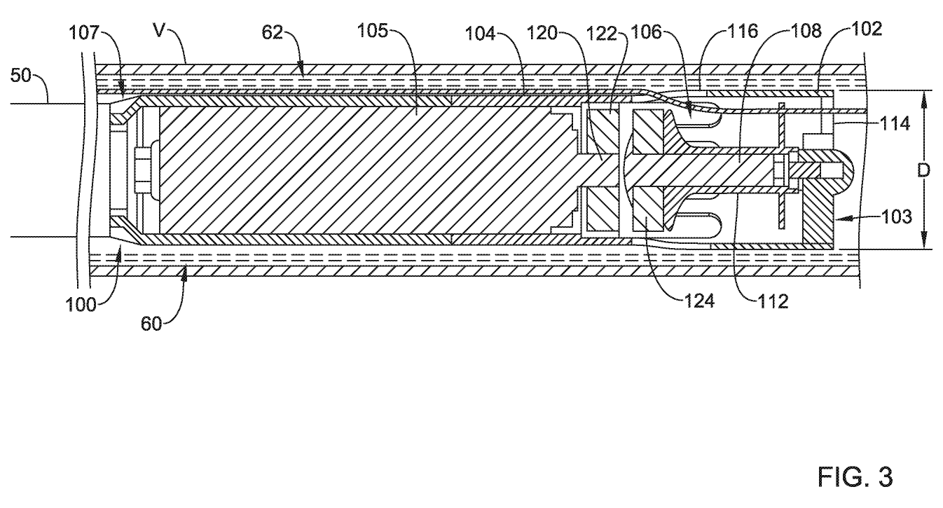

FIG. 3 depicts a schematic cross-section view of a body portion 62 of a delivery or guide sheath 60 (hereinafter “delivery sheath 60”) extending into a blood vessel V with the blood pump 100 inserted into the delivery sheath 60. The blood pump 100 may include and/or may be coupled with the elongate shaft 50 extending proximally from a proximal end 107 of the blood pump 100 to a location outside of the blood vessel V and the delivery sheath 60. When positioning the blood pump 100 within the patient, the blood pump 100 may be advanced through the blood vessel V and positioned in or at a target location, such as a target cardiac location (e.g., in the aorta 22, across the aortic valve 20, and/or into the left ventricle 16), via the delivery sheath 60. While the delivery sheath 60 is illustrated in FIG. 3 with the use of the blood pump 100, various other medical devices may be used in conjunction with the delivery sheath 60.

As depicted in FIG. 3, a distal end of the elongate shaft 50 may be coupled with the proximal end 107 of the blood pump 100 in any suitable manner. For example, the elongate shaft 50 may be fixedly coupled with the blood pump 100 or releasably coupled with the blood pump 100. When the elongate shaft 50 is fixedly coupled with the blood pump 100, the elongate shaft 50 may be mechanically and/or electrically coupled to the blood pump 100 using any suitable technique including, but not limited to, welding, soldering, molding, over-molding, re-flowing, friction fittings, and/or other suitable coupling techniques. When the elongate shaft 50 is releasably coupled with the blood pump 100, the elongate shaft 50 may be mechanically and/or electrically coupled to the blood pump 100 using any suitable technique including, but not limited to, threads, magnets, inductive components, friction fittings, ball-detent connections, and/or other suitable couplings.

The blood pump 100 may include one or more components. In some examples, the blood pump 100 may include an impeller housing 102 and a motor housing 104. The impeller housing 102 and the motor housing 104 may be integrally or monolithically constructed. Alternatively, the impeller housing 102 and the motor housing 104 may be separate components. The impeller housing 102 may carry an impeller assembly 106 therein. The impeller assembly 106 may include an impeller shaft 108 and an impeller 112 that rotates relative to the impeller housing 102 to drive or pump blood from the left ventricle 16 of the heart 18 of the patient (e.g., via the cannula 40 of or in fluid communication with the blood pump 100) to the aorta 22 or portion of the anatomy in communication with the aorta (e.g., from the pump blood 100 through the blood pump 100 to an ascending aorta). In some examples, the impeller shaft 108 and the impeller 112 may be integrally formed, whereas, in other examples the impeller shaft 108 and the impeller 112 may be separate components.

The blood pump 100 may have any suitable outer diameter D, which may be defined by the impeller housing 102, the motor housing 104, and/or other suitable component forming a widest dimension along a length of the blood pump 100. In some examples, the blood pump 100 may be classified as being a large-bore blood pump 100 or a small-bore blood pump 100, where the large-bore blood pump 100 may have an outer diameter D of 7 millimeters (mm) (0.276 inches) or greater and the small-bore blood pump 100 may have an outer diameter D of less than 7 mm. In one example, a large-bore blood pump 100 may have an outer diameter of at least 8 mm (0.315 inches). In one example, a large-bore blood pump 100 may have an outer diameter of 8 mm, but other suitable outer diameters are contemplated.

Rotation of the impeller 112 may cause blood to flow from a blood inlet 114 formed on the impeller housing 102 (e.g., where the blood may enter the blood inlet 114 directly from the left ventricle and/or via the cannula 40), through the impeller housing 102, and out of a blood outlet 116 formed on the impeller housing 102. As shown in FIG. 3, the blood inlet 114 may be formed on an end portion of the impeller housing 102 and the blood outlet 116 may be formed on a side portion of the impeller housing 102. In other examples, the blood inlet 114 and/or the blood outlet 116 may be formed on or at other portions of the impeller housing 102 or other components of the system 10.

A size of the impeller 112 may be determined based on the outer diameter D of the blood pump 100. For example, a blood pump 100 configured with a larger outer diameter may facilitate use of a larger impeller 112 and a blood pump 100 configured with a smaller outer diameter may require a smaller impeller 112. In some examples, when the blood pump 100 is configured to have a larger impeller 112, the blood pump 100 may be able to achieve a desired flow rate of blood through the blood pump at lower rotations per minute (RPM) than a smaller impeller 112 would require to achieve the desired flow rate.

The impeller housing 102 of the impeller assembly 106 may be coupled to the cannula 40 and the cannula 40 may extend distally from the impeller housing 102 (e.g., extending distally from the distal end 103 of the blood pump 100), and the cannula 40 may receive blood from the left ventricle and deliver the blood to the blood inlet 114. Alternatively or additionally, the impeller housing 102 may be further elongated than depicted in FIG. 3 and define all of or at least part of the cannula 40 extending into the left ventricle of the patient. Other suitable configurations of the impeller housing 102 and/or the cannula 40 are contemplated.

The motor housing 104 may carry a motor 105, and the motor 105 may be configured to rotatably drive the impeller 112 relative to the impeller housing 102 when the motor 105 is actuated. In some examples, the motor 105 may rotate a drive shaft 120 coupled with a driving magnet 122. Rotation of the driving magnet 122 may cause rotation of a driven magnet 124 that may be coupled with and/or may be part of the impeller assembly 106. More specifically, in configurations incorporating the impeller shaft 108, the impeller shaft 108 and the impeller 112 may be configured to rotate with the driven magnet 124. In other configurations, the motor 105 and/or the drive shaft 120 may be coupled to the impeller assembly 106 directly and/or via other suitable components.

As discussed, the blood pump 100 may be delivered to the heart of the patient via an arterial access site. In some examples, the blood pump 100 may be delivered to the heart of the patient via a femoral artery access site, a femoral artery, and the aorta of the patient. However, utilizing a femoral artery access site may limit the mobility of the patient as movement of the elongate shaft 50 as the patient moves may result in inadvertent adjustment of a position of the blood pump 100 in the heart of the patient. As such, to allow the patient to be ambulatory with the blood pump 100 operating in the heart of the patient (e.g., with the blood pump 100 providing cardiac assistance), it may be advantageous to utilize an access site closer to the heart of the patient and/or not at a limb of the body that moves relative to a core (e.g., relative to a chest) of the patient as the patient becomes ambulatory. In some examples, the axillary artery or subscapular artery may have a sufficient diameter to receive a small-bore blood pump and is close enough to the heart of the patient (e.g., located at the chest of the patient) to mitigate a likelihood of the elongate shaft 50 adjusting in a manner that would inadvertently adjust the position of the blood pump 100 in the heart of the patient as the patient becomes ambulatory.

To treat heart failure and/or other cardiac diseases, the blood pump 100 may provide cardiac assistance by helping to achieve a desired flow rate out of the heart 18 and into the arterial system, while reducing a required workload on the heart 18. When a patient is in cardiogenic shock or is recovering from cardiogenic shock, the patient may require left ventricular support to ensure sufficient oxygenated blood reaches the heart 18 and/or other organs for recovery from the cardiogenic shock. To support a patient in or recovering from cardiogenic shock, the blood pump 100 may be required to achieve a flow rate of 5.5 liters per minute (L/min) or to ensure blood is leaving the left ventricle 16 at a rate of 5.5 L/min. Further, as the patient is recovering from cardiogenic shock and while the blood pump 100 is positioned within and operating in the patient, the patient should be ambulatory.

To facilitate allowing the patient to be ambulatory, an axillary artery access site for the delivery and operation of the blood pump 100 may be more optimal than a femoral artery access site, as discussed. However, to utilize an axillary artery access site for delivering the blood pump 100 to the heart 18 of the patient, the blood pump 100 must be a small-bore (e.g., a low profile) blood pump 100. To achieve the desired flow rate of blood (e.g., 5.5 L/min) out of the heart 18 when the patient is in or is recovering from cardiogenic shock, the small-bore blood pump 100 may have to operate at a higher RPM than a large-bore blood pump 100 due to having a smaller impeller 112 than the large-bore blood pump 100. Pumping blood with a higher RPM to achieve the desired flow rate may result in an increase in likelihood of causing hemolysis (e.g., the destruction of blood cells due to shear force) relative to when the desired flow rate is achieved with a lower RPM. Hemolysis can lead to acute kidney damage, which can develop into chronic kidney disease, and/or cause thrombus formation, which can increase a risk of ischemic stroke. As a result, it is desirable to mitigate hemolysis.

The concepts disclosed herein may facilitate the use of a large-bore blood pump 100 to provide cardiac assistance, while allowing the patient with the blood pump 100 positioned in or at the heart 18 to be ambulatory. For example, the large-bore blood pump 100 may be delivered to the heart 18 or proximate to the heart 18 of the patient through a femoral artery access site and the blood pump 100 may be operated via a cable (e.g., the elongate shaft 50) extending through an axillary artery access site, which facilitates achieving a desired flow rate of blood from the heart 18 of the patient and mitigating hemolysis, while allowing the patient to be ambulatory when the blood pump 100 is positioned and operating in the heart 18.

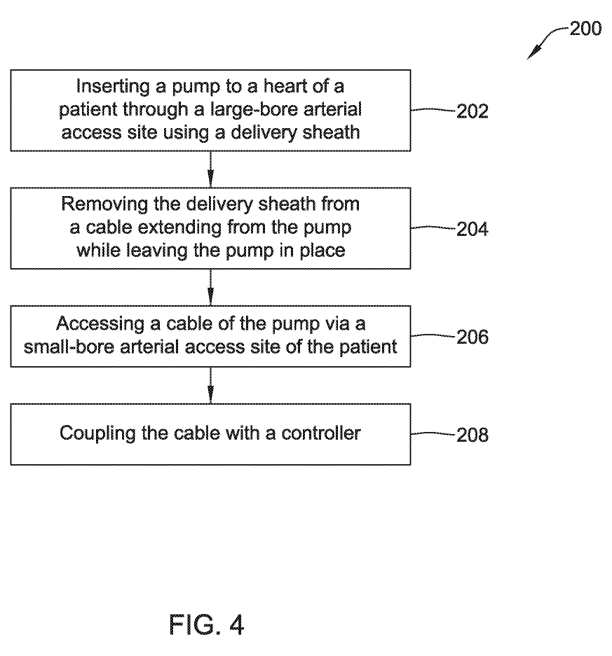

FIG. 4 depicts a schematic diagram of an illustrative method 200 of positioning and operating a blood pump at or proximate a heart of a patient. The method 200 may include the blood pump configurations discussed herein and/or other suitable blood pump configurations.

The method 200 may include inserting 202 a blood pump to a heart of a patient through a large-bore arterial access site. In some examples, the large-bore arterial access site may be configured to receive a blood pump with an outer profile or diameter of at least 7 mm, where the blood pump is configured to achieve or facilitate achieving a flow rate of blood from the heart of the patient of at least 5.5 L/min. In some examples, the large-bore arterial access site may be a femoral artery access site, but other suitable large-bore arterial access sites are contemplated.

The blood pump may be inserted into a heart of the patient in any suitable manner. In some examples, a delivery sheath may be inserted through the large-bore arterial access site, into the arterial system, and to a location in an aorta of the patient at or proximate the heart of the patient (e.g., a target location or site at or proximate an aortic valve of the patient). In some examples, the delivery sheath may be delivered to the location at or proximate the heart over a guidewire previously delivered to the location via the large-bore arterial access site, but other suitable configurations for delivering the delivery sheath are contemplated. With the delivery sheath positioned at or proximate the heart, the blood pump may be delivered through the delivery sheath to the heart or a location proximate the heart of the patient.

Once the blood pump has been delivered to or proximate the heart of the patient, the delivery sheath may be removed 204 from a cable (e.g., an elongate shaft) extending from the blood pump while leaving the blood pump in place. In some examples, the delivery sheath may be removed from the cable extending from the blood pump by advancing the delivery sheath in a proximal direction through the large-bore arterial access site. Although the delivery sheath may be removed from the cable and/or patient in some examples, in other examples the delivery sheath or other sheath may remain within the patient to facilitate re-engaging the blood pump at a later time and/or for other suitable purposes. In some examples, a smaller bore sheath may replace the delivery sheath across the arterial access site to facilitate inserting medical devices into the vasculature of the patient at a later time without unduly blocking blood flow through the femoral artery or other artery proximate the large-bore arterial access site.

The cable may have any suitable length. In some examples, the cable may be shorter than a distance from the femoral artery access site or other suitable large-bore arterial access site to the heart, but long enough to extend from the heart of the patient through an axillary artery access site or other suitable small-bore arterial access site. In some examples, once the delivery sheath has been removed from the cable, the cable may extend through the aorta with a loose proximal end in the aorta.

Once the delivery sheath has been removed from the cable extending from the blood pump, the cable may be accessed 206 via a small-bore arterial access site. For example, a grasping device may be inserted into the arterial vasculature of the patient through a small-bore arterial access site nearer the heart than the large-bore arterial access site, the cable may be grasped with the grasping device, and the cable may be pulled through the small-bore arterial access site. In some examples, the small-bore arterial access site may be an axillary artery access site, a sub-scapular artery access site, and/or one or more other suitable arterial access sites in communication with the aorta of the patient, proximate the heart of the patient, and which may facilitate allowing the patient to be ambulatory while the blood pump is operating within the patient.

In operation, a user may insert the grasping device through the small-bore arterial access site, through the arterial vasculature, to the aorta (e.g., the ascending aorta, the aortic arch, and/or the descending aorta) in which the cable is extending after delivery of the blood pump to the heart of the patient. With the grasping device, the user may grasp the cable, pull the cable through the aorta into an artery directly in communication with the small-bore arterial access site, and through the small-bore arterial access site. In some example, the grasping device may be a snare, a forceps, and/or other suitable grasping device. In one example, the grasping device may be a retrieval snare.

Once a proximal end of the cable has been extended through the small-bore arterial access site, the cable may be coupled 208 with a controller. In some examples, the cable may be and/or may include one or more of a power cable, control cable, a wire, a coaxial cable, an optical cable, and/or other suitable cable. The controller may be configured to power and/or control operation of the blood pump. In some examples, the controller may be configured to couple to the patient, be held by the patient, and/or be worn by the patient, such that the patient may be ambulatory while the cable is coupled with the controller. Once the cable is coupled with the controller, operation of the blood pump may be initiated (e.g., via the controller) to provide a flow rate of blood through the blood pump (e.g., 5.5 L/min) that is sufficient to treat and/or facilitate recovering from cardiogenic shock or other cardiac condition, while mitigating hemolysis and allowing the patient to be ambulatory.

The cable may be coupled with the controller in any suitable manner. In some examples, the cable may be coupled with the controller using a mechanical plug, an electrical plug, an induction coupling, a magnetic coupling, a threaded coupling, and/or one or more other suitable type of coupling.

The controller may be any suitable type of controller and/or control module. For example, the controller may be and/or may include any suitable computing device configured to process data for, provide power to, and/or control operation of the blood pump. The controller may include a user interface, but other suitable configurations of the controller are contemplated.

The controller may be configured to monitor and/or control an amount of power and/or a frequency of power applied to blood pump according to a preconfigured control program, according to user interactions with the controller, and/or in response to values from one or more sensors and/or values of one or more monitored metrics or parameters reaching or going beyond a threshold value. In some examples, the controller may be configured to provide electrical current, voltage pulses, optical pulses, and/or other suitable pulses of power to the blood pump to operate a motor and impeller of the blood pump, but other suitable configurations are contemplated. The controller may include or be in communication with a power source, such as a wall power source, a battery power source, a renewable energy power source, and/or other suitable source of power.

The illustrative controller may include, among other suitable components, one or more processors, memory, one or more power modules, one or more input/output (I/O) units, and/or other suitable components. Example other suitable components of the controller may include, but are not limited to, communication components, a touch screen, selectable buttons, a housing, and/or other suitable components of a controller.

The processor of the controller may include a single processor or more than one processor working individually or with one another. The processor may be configured to receive and execute instructions, including instructions that may be loaded into the memory and/or other suitable memory. Example components of the processor may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory of the controller may include a single memory component or more than one memory component each working individually or with one another. Example types of memory may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory may be or may include a transitory or non-transitory computer readable medium. The memory may include instructions stored in a transitory and/or non-transitory state on a computer readable medium that may be executable by the processor to cause the processor to perform one or more of the methods and/or techniques described herein.

The power module, when included, may include any suitable component configured to facilitate providing power to the blood pump. Example suitable components of the power module may include, but are not limited to, a DC-DC converter, a DC-AC converter, an optical converter (e.g., a component configured to convert electrical power to into optical power (e.g., a light or laser beam)), a high voltage capacitor, a transistor switch, a power measurement unit (e.g., voltage measurement unit, current measurement unit, optical power measurement unit, and/or other suitable measurement unit), a device identification unit (e.g., a unit that identifies whether the medical device is compatible with the controller), and/or other suitable components.

The I/O units of the controller may include a single I/O component or more than one I/O component each working individually or with one another. Example I/O units may be or may include any suitable types of mechanical communication hardware, electrical communication hardware, optical communication hardware, and/or software including, but not limited to, power input ports to receive power from a power source, power output ports to provide power to the blood pump, device ports for coupling with the blood pump, communication ports configured to communicate with electronic components of the blood pump and/or with other suitable computing devices or systems. Example types of I/O units may include, but are not limited to, wired power components, wired optical components, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless power components, wireless optical components, wireless communication components (e.g., radio frequency (RF) components, Low-Energy BLUETOOTH protocol components, BLUETOOTH protocol components, Near-Field Communication (NFC) protocol components, WI-FI protocol components, optical communication components, ZIGBEE protocol components, and/or other suitable wireless communication components), and/or other suitable I/O units.

The user interface, when included, may be configured to communicate with the controller via one or more wired or wireless connections. The user interface may include one or more display devices, one or more input devices, one or more output devices, and/or one or more other suitable features.

The display device may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s) may be and/or may include any suitable components and/or features for receiving user input via the user interface. Example input device(s) 42 include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components), and/or other suitable input devices.

The output device(s) may be and/or may include any suitable components and/or features for providing information and/or data to users and/or other computing components. Example output device(s) include, but are not limited to, displays, speakers, vibration systems, tactile feedback systems, optical outputs, cables, lights, and/or other suitable output devices.

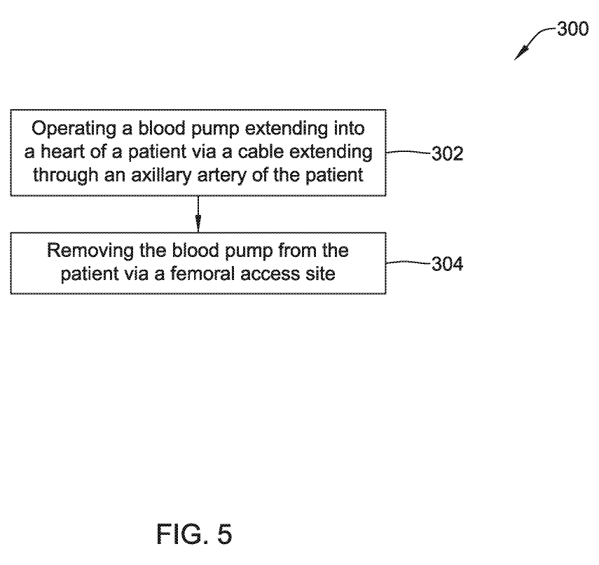

FIG. 5 schematically depicts an illustrative method 300 of using a blood pump. The method 300 may include operating 302 a blood pump (e.g., a large-bore blood pump) extending into a heart of a patient via a cable (e.g., an elongate shaft) extending through an axillary artery of the patient. In some examples, the cable extending through the axillary artery may have a distal end coupled with the blood pump and a proximal end that has been extended through the axillary artery and an arterial access site at or in communication with the axillary artery. In some examples, the blood pump may be a large-bore blood pump having an outer diameter equal to or greater than an outer diameter of an artery at the arterial access site through which the cable extends.

After use of the blood pump and/or at one or more other suitable times for removal of the blood pump from the patient, the blood pump may be removed 304 from the patient via a femoral artery access site. In some examples, the blood pump may be a large-bore blood pump and the femoral artery, along with the femoral artery access site, may be able to accommodate the outer diameter of the large-bore blood pump passing therethrough.

The blood pump may be removed from the patient through the femoral artery access site and/or other large-bore arterial access site in any suitable manner. For example, the blood pump may be removed from the patient with the cable attached thereto or the blood pump may be removed from the patient with the cable detached from the blood pump prior to removal of the blood pump from the patient. Other suitable configurations are contemplated for removing the blood from the patient.

When removing the blood pump from the patient with the cable attached to the blood pump, a user may detach a proximal end of the cable from the controller. Further, the user may insert a removable sheath into the aorta of the patient via the femoral artery access site and the femoral artery or via one or more other large-bore arterial access site. The retrieval sheath may be similar to or different than a delivery sheath and may include a grasping device at a distal end. Any suitable grasping device may be utilized including, but not limited to, a snare, a forceps, and/or other suitable grasping device. In one example, the grasping device extending from distal end of the retrieval sheath may be a snare (e.g., a retrieval snare) that may be operable from a proximal end of the retrieval sheath, exterior the patient.

With the cable grasped by the grasping device, the cable may be pulled out of the axillary artery or other suitable artery in communication with the aorta and positioned in the aorta. With the cable positioned in the aorta, the retrieval sheath may extend over and/or be coupled with or otherwise cover an entirety of or at least a portion of the blood pump for removal of the blood pump from the heart. The retrieval sheath may extend over and/or couple with the blood pump in any suitable manner. For example, the retrieval sheath may couple with the blood pump by extending over the blood pump, via the grasping device, via a threaded connection, via a magnetic connection, and/or other suitable coupling techniques. Once the retrieval sheath is coupled with the blood pump, the blood pump may be removed from the patient via the femoral artery access site with the retrieval sheath. Alternatively or additionally, the grasping device may be configured to grasp the cable and/or the blood pump. The grasping device may then be configured to pull the blood pump into the retrieval sheath and through the retrieval sheath and out of a proximal end of the retrieval sheath. Then, the retrieval sheath may be removed from the femoral artery access site or other suitable large-bore arterial access site by advancing the retrieval sheath in the proximal direction.

When removing the blood pump from the patient with the blood pump detached from the cable, a user may detach the cable from the blood pump and remove the cable from the vasculature of the patient via the axillary artery access site or other suitable small-bore arterial access site. The cable may be detached or separated from the blood pump in any suitable manner. In an example, a threaded connection may be disconnected by rotating the cable relative to the blood pump. In another example, a magnetic coupling between the cable and the blood pump may be released via activation of a button or actuator at the controller. In a further example, a mechanical coupling between the cable and the blood pump may be released via activation of a button or actuator at the controller. In another example, a coupling between the cable and the blood pump may be released in response to the retrieval sheath interacting with the cable and/or the blood pump, and/or the cable and the blood pump may be separated in one or more other suitable manners.

Once the blood pump has been separated from the cable, the blood pump may be retrieved from the heart of the patient and removed from the patient. If a retrieval sheath is not already inserted into the vasculature via the femoral artery access site or other large-bore arterial access site, the retrieval sheath may be inserted into the aorta to a location of or proximate the blood pump. The retrieval sheath may couple with or otherwise cover an entirety of or at least a portion of the blood pump for removal of the blood pump from the heart. The retrieval sheath may couple with the blood pump in any suitable manner. For example, the retrieval sheath may couple with the blood pump by covering the blood pump via a grasping device of the retrieval sheath, via a threaded connection, via a magnetic connection, and/or other suitable coupling techniques. Once the retrieval sheath is coupled with the blood pump, the blood pump may be removed from the patient via the femoral artery access site with the retrieval sheath. Alternatively, a grasping device may be configured to grasp the blood pump. The grasping device may then be configured to pull the blood pump into the retrieval sheath and through the retrieval sheath and out of a proximal end of the retrieval sheath. Then, the retrieval sheath may be removed from the femoral artery access site or other suitable large-bore arterial access site by advancing the retrieval sheath in the proximal direction.

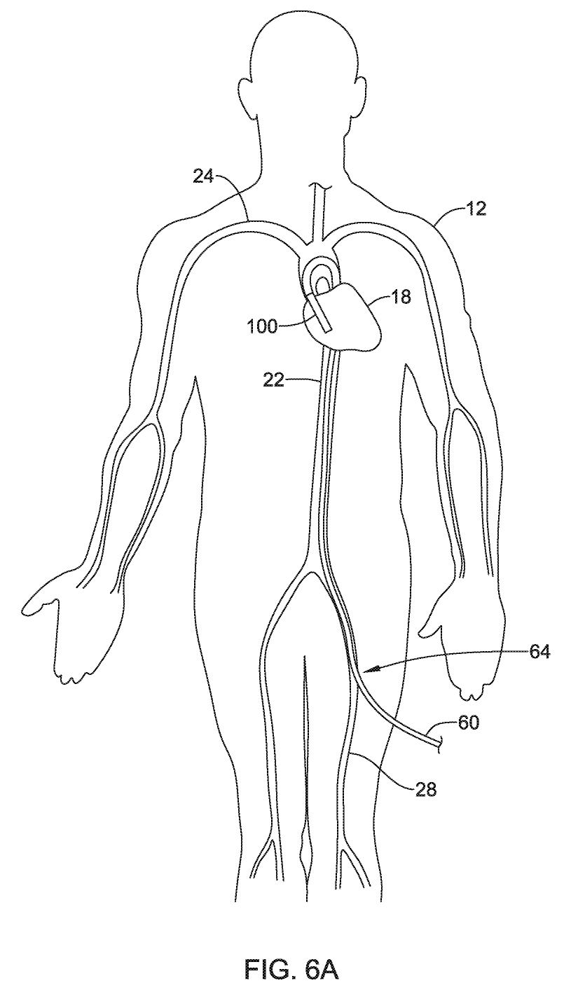

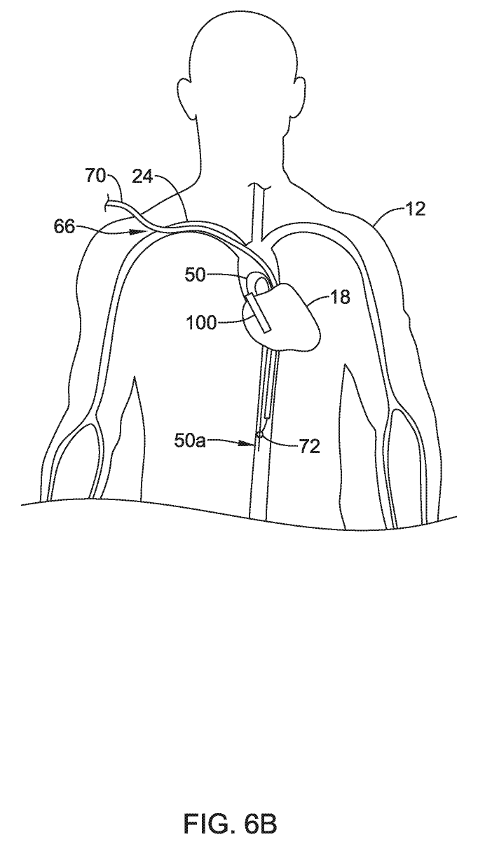

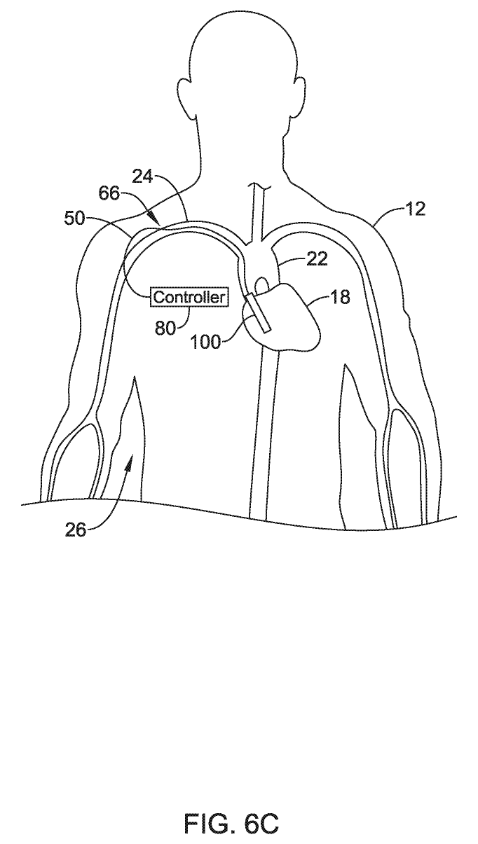

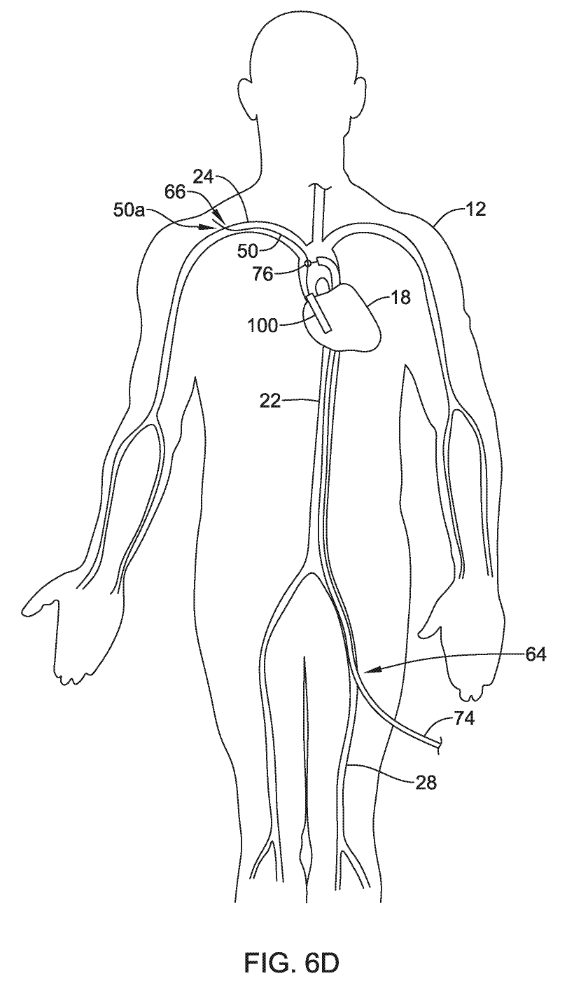

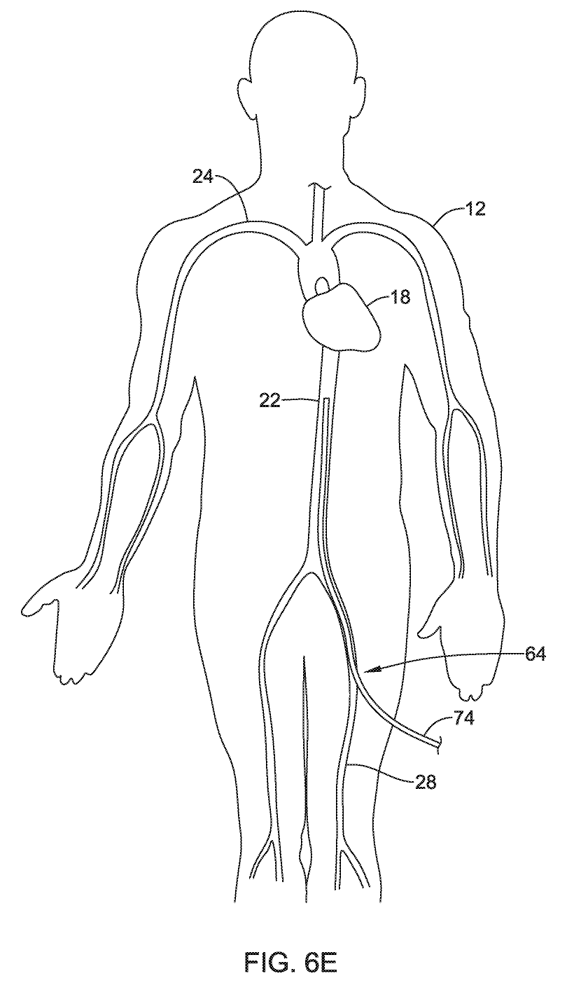

FIGS. 6A-6E depict schematic diagrams of a technique for using the blood pump 100 (e.g., for positioning the blood pump 100 (e.g., a large-bore blood pump) in the heart 18 of a patient 12, operating the blood pump 100 in a manner that allows the patient 12 to be ambulatory, and removing the blood pump 100 from the patient 12). Although a femoral artery access site 64 and an axillary artery access site 66 are utilized in FIGS. 6A-6E, other suitable large-bore arterial access sites and/or small-bore arterial access sites, respectively, may be utilized.

FIG. 6A depicts a schematic diagram of the patient 12 with a delivery sheath 60 coupled with and used to deliver the blood pump 100 to the heart 18 of the patient 12. The blood pump 100 may be delivered to the heart 18 of the patient 12 with the delivery sheath 60 or the delivery sheath 60 may be positioned in the aorta 22 proximate the heart 18 and then the blood pump 100 may be delivered through delivery sheath using a delivery tool or shaft. An axillary artery 24 of the patient may extend from the aorta 22.

As depicted in FIG. 6A, the blood pump 100 may be delivered to the heart 18 with a distal end of the blood pump 100 in the left ventricle of the heart 18 and with a proximal end of the blood pump 100 positioned in the aorta such that the blood pump 100 crosses the aortic valve (the aortic valve is not labeled in FIG. 6A). Once the blood pump 100 is positioned at the heart 18, the delivery sheath 60 may be removed from the patient 12 (e.g., removed from the blood pump 100, the elongate shaft 50, and the vasculature of the patient 12) via a femoral artery 28 and the femoral artery access site 64 while leaving the blood pump 100 in place.

FIG. 6B depicts a schematic diagram of the patient 12 with the blood pump 100 positioned at the heart 18 of the patient and the elongate shaft 50 (e.g., a cable) extending into the aorta 22 and a proximal end 50a of the elongate shaft 50 loosely extending within the aorta 22 (e.g., within the descending aorta). To facilitate coupling the elongate shaft 50 with a controller, a user may insert a grasping tool 70 into the axillary artery 24 via the axillary artery access site 66. The grasping tool 70 may be inserted through the axillary artery 24, into the aorta 22 proximate the elongate shaft 50. In some examples, the grasping tool 70 may be a snare 72 and a proximal end 50a of the elongate shaft 50 may be threaded through the snare 72 and the snare 72 may be actuated to tighten around the elongate shaft 50 such that the grasping tool is grasping the elongate shaft 50. Although the grasping tool 70 is depicted as including the snare 72, the grasping tool 70 may include one or more other suitable grasping components including, but not limited to, a forceps. Once the elongate shaft 50 has been grasped with the grasping tool 70, the grasping tool 70 may be advanced in a proximal direction through the aorta 22, the axillary artery 24, and the axillary artery access site 66 such that the elongate shaft 50 may extend or be positioned exterior of the patient 12 for connection to a controller and/or suitable operating component.

FIG. 6C depicts a schematic diagram of the patient 12 with the elongate shaft 50 extending from the blood pump 100 positioned at the heart 18 of the patient 12 with the elongate shaft 50 extending through the axillary artery access site 66 and coupled with a controller 80. The controller 80, once coupled with the elongate shaft 50, may be configured to supply power to the blood pump 100, send control signals to the motor of the blood pump 100, and/or receive data from sensors at the blood pump 100.

In some examples, the elongate shaft 50 and/or the controller 80 may be configured to facilitate operating the blood pump 100 while the patient is ambulatory. In some examples, the elongate shaft 50 may have a length sufficient to secure the controller 80 with the elongate shaft 50 connected thereto relative to a core 26 of the patient 12 such that the controller 80 and the elongate shaft 50 may be positioned to mitigate inadvertent movements of the blood pump 100 within the patient. In some examples, the controller 80 may be secured to a chest or back of the patient 12 using any suitable securing mechanism including, but not limited to, adhesive, suction, one or more straps, compression, and/or other suitable securing mechanisms or techniques. To further mitigate movement of the elongate shaft 50 and/or the controller 80 and thus, movement of the blood pump 100, the axillary artery access site 66 may be closed around the elongate shaft 50. Additionally, closing the axillary artery access site 66 around the elongate shaft 50 may mitigate bleeding from the axillary artery access site 66 and a likelihood of infection at or through the axillary artery access site 66.

Once the patient 12 has recovered, the blood pump 100 may be removed from the patient 12. FIG. 6D depicts a schematic diagram of the patient 12 with the elongate tube disconnected from the controller 80 (not depicted in FIG. 6D) and the controller 80 removed from the patient 12. When disconnected from the controller 80, the proximal end 50a of the elongate shaft 50 may be freely located outside of the axillary artery access site 66. If the axillary artery access site 66 was closed, the axillary artery access site 66 may be reopened to allow the elongate shaft 50 to be advanced through the axillary artery access site 66 into the axillary artery 24 and the aorta 22.

A retrieval sheath 74 be inserted through the femoral artery access site 64, through the femoral artery 28, and into the aorta 22 to a location proximate the elongate shaft 50 extending into the axillary artery 24. In some examples, the retrieval sheath 74 may include or may be used with a snare 76 or other suitable grasping tool to grasp the elongate shaft 50 and pull the elongate shaft 50 through the axillary artery access site 66, through the axillary artery 24, through the aorta 22, and into the retrieval sheath 74. In some examples, advancing the snare 76 in a proximal direction may advance the blood pump 100 out of the heart of the patient and into the retrieval sheath 74 for removal.

FIG. 6E depicts a schematic diagram with the blood pump 100 (not shown in FIG. 6E) removed from the heart 18 with the retrieval sheath 74 advanced in a proximal direction for removal from the vasculature of the patient 12. The blood pump 100 may be retrieved and removed from the patient 12 with the retrieval sheath 74 positioned at or proximate the heart 18 of the patient 12. Alternatively, once the blood pump 100 is entirely or at least partially positioned within the retrieval sheath 74, the retrieval sheath 74 and the blood pump 100 may be advanced in the proximal direction and removed from the patient 12 together via the femoral artery 28 and the femoral artery access site 64.

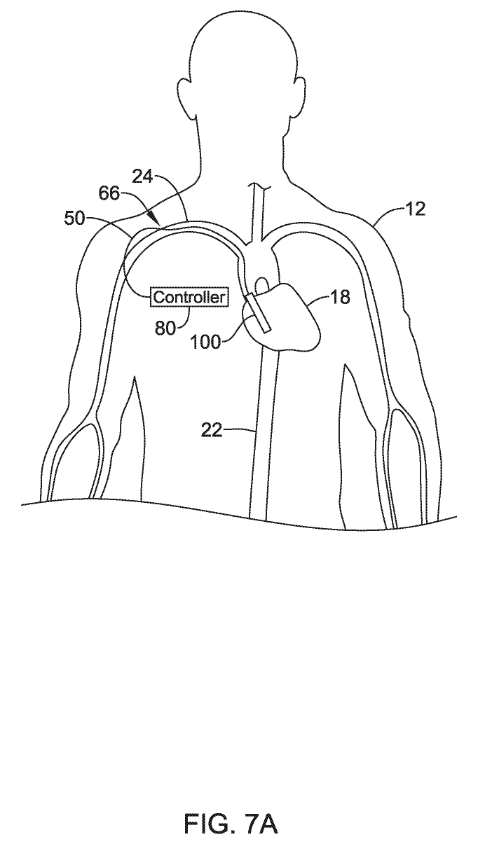

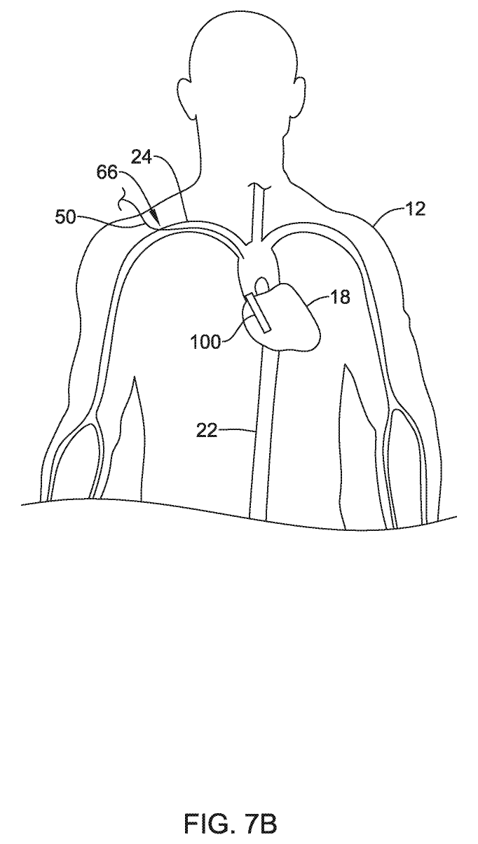

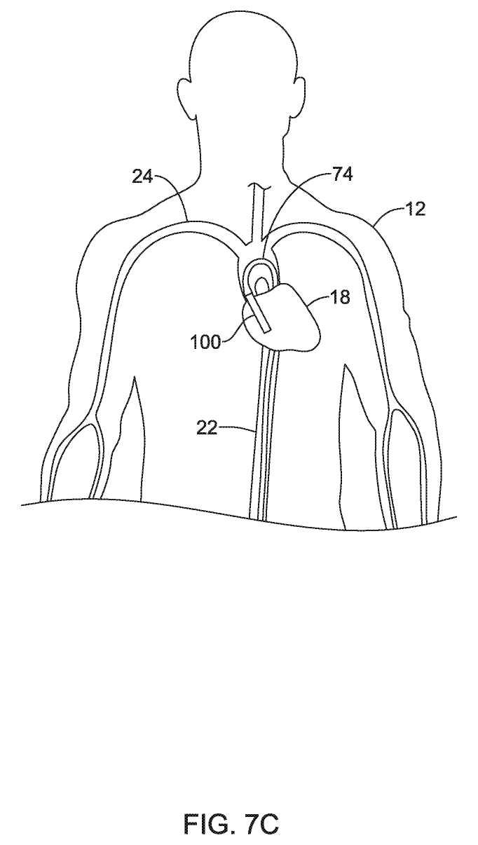

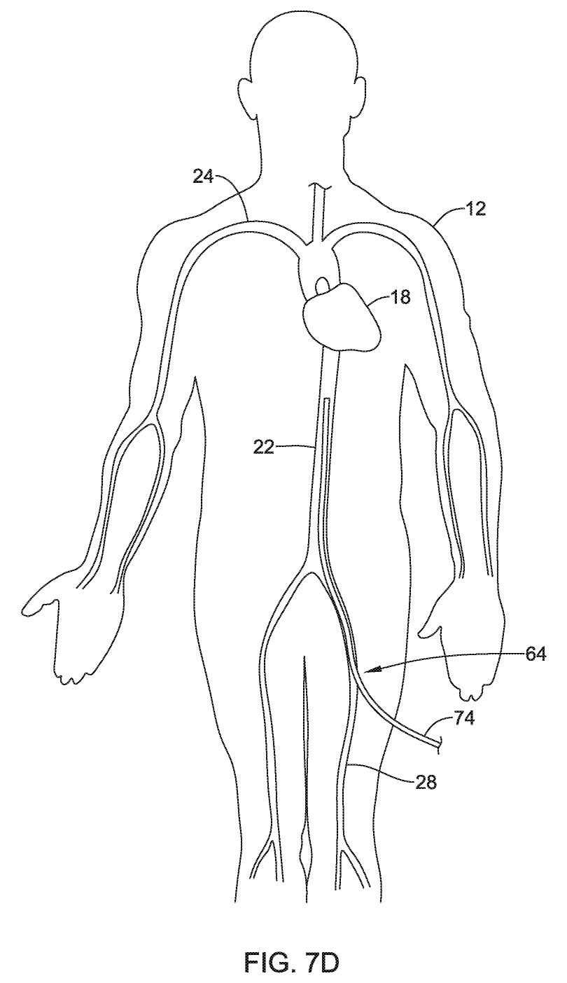

FIGS. 7A-7D depict schematic diagrams of a technique for using the blood pump 100 (e.g., a large-bore blood pump) in the heart 18 of a patient 12 and removing the blood pump 100 from the patient 12. Although the femoral artery access site 64 and the axillary artery access site 66 are utilized in FIGS. 7A-7E, other suitable large-bore arterial access sites and/or small-bore arterial access sites, respectively, may be utilized.

FIG. 7A depicts a schematic diagram of the patient 12 with the elongate shaft 50 extending from the blood pump 100 positioned at the heart 18 of the patient 12, through the axillary artery 24, through the axillary artery access site 66, and coupled with the controller 80. The controller 80, once coupled with the elongate shaft 50, may be configured to supply power to the blood pump 100, send control signals to the motor of the blood pump 100, and/or receive data from sensors at the blood pump 100. The controller 80 may be coupled with the patient 12 in any suitable manner including, but not limited to, as discussed herein with respect to FIG. 6C. The positioning of the elongate shaft 50 and the controller 80 depicted in FIG. 7A may facilitate use of the blood pump 100 within the patient while the patient 12 is ambulatory.

Once the patient 12 has recovered from a cardiac condition being treated, the blood pump 100 may be removed from the patient 12. In some examples, the elongate shaft 50 may be removed from the blood pump 100 to facilitate removing the blood pump 100 from the patient 12. FIG. 7B depicts a schematic diagram of the patient 12 with the elongate shaft 50 disconnected from the controller 80 (not depicted in FIG. 7B), the controller 80 removed from the patient 12, and the elongate shaft 50 disconnected from the blood pump 100 positioned in the heart 18 of the patient 12.

The elongate shaft 50 may be disconnected from the blood pump 100 in any suitable manner. For example, the elongate shaft 50 may be disconnected from the blood pump 100 by rotating the elongate shaft 50 to disconnect a threaded connection, by actuating a button at the controller 80 to disconnect a magnetic connection between the elongate shaft 50 and the blood pump 100, by actuating a button at the controller 80 to disconnect a mechanical connection (e.g., a latch, etc.) between the elongate shaft 50 and the blood pump 100, and/or the elongate shaft 50 may be disconnected from the blood pump 100 in one or more other suitable manners. Once disconnected from the blood pump 100, the elongate shaft 50 may be advanced through the axillary artery 24 and the axillary artery access site 66 to remove the elongate shaft 50 from the vasculature of the patient. After removing the elongate shaft 50 from the axillary artery access site 66, the axillary artery access site may be closed.

FIG. 7C depicts a schematic diagram of the retrieval sheath 74 inserted into the aorta 22 (e.g., through the femoral artery access site 64, not shown in FIG. 7C) and to a location of or proximate a proximal end of the blood pump 100. Once at the blood pump 100, the retrieval sheath 74 may couple with and/or grasp the blood pump 100 to withdraw the blood pump 100 from the heart 18 of the patient 12. The retrieval sheath 74 may include a snare, a magnetic connector, a threaded connector, and/or other suitable connector configured to couple with the blood pump 100 for removal of the blood pump 100 from the patient. Although other suitable configurations are contemplated, the retrieval sheath 74 may be configured to couple with one or more components of the blood pump 100 with which the elongate shaft 50 was coupled. Once the retrieval sheath 74 is coupled with the blood pump 100, the retrieval sheath 74 and the blood pump 100 may be removed from the vasculature of the patient.

FIG. 7D depicts a schematic diagram with the blood pump 100 (not shown in FIG. 7D) removed from the heart 18 with the retrieval sheath 74 advanced in a proximal direction for removal from the vasculature of the patient 12. The blood pump 100 may be retrieved and removed from the patient 12 with the retrieval sheath positioned at or proximate the heart 18 of the patient 12. Alternatively, once the blood pump 100 is entirely or at least partially positioned within the retrieval sheath 74, the retrieval sheath 74 and the blood pump 100 may be advanced in the proximal direction and removed from the patient 12 together via the femoral artery 28 and the femoral artery access site 64.

The materials that can be used for the various components of the devices, assembly, systems, and the various elements thereof disclosed herein may include those commonly associated with medical devices. In some instances, the devices described herein, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the devices, assemblies, and/or systems described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility may be imparted into the devices and/or other elements disclosed herein. For example, the devices described herein, and/or components or portions thereof, may be made of a material that does not substantially distort the image and create substantial artifacts (e.g., gaps in the image). Certain ferromagnetic materials, for example, may not be suitable because they may create artifacts in an MRI image. The devices, assembly, and/or systems described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The scope of the disclosure is, of course, defined in the language in which the appended claims are expressed.

What is claimed is:

1. A mechanical circulatory support system comprising:
a blood pump configured to extend into a ventricle of a heart of a patient; and
an elongate shaft coupled to the blood pump and configured to be pulled through an aorta of the patient and into an axillary artery of the patient while the blood pump is in the heart of the patient.

2. The system of claim 1, wherein the blood pump has an outer diameter of at least 8 millimeters (mm) and is configured to achieve a flow rate of at least 5.5 liters per minute (L/min).

3. The system of claim 1, wherein the elongate shaft is detachable from the blood pump and is configured to be removed from the patient through the axillary artery.

4. The system of claim 1, wherein the elongate shaft has a length sufficient to extend from the blood pump in the ventricle of the heart of the patient to an access site through the axillary artery.

5. The system of claim 1, wherein the elongate shaft has a length that is shorter than a distance from a femoral artery access site, through the aorta, and to a location in the aorta proximate the heart of the patient.

6. The system of claim 1, further comprising:
a power source configured to releasably couple with the elongate shaft.

7. The system of claim 1, further comprising:
a controller configured to couple with the elongate shaft and control operation of the blood pump, and
wherein the controller is configured to be secured to a body of the patient.

8. The system of claim 1, further comprising:
a retrieval snare configured to access the elongate shaft in the aorta of the patient and move the elongate shaft through the axillary artery.

9. The system of claim 1, further comprising:
a delivery sheath configured to receive the blood pump and the elongate shaft, and
wherein the delivery sheath is configured to extend from a femoral artery access site, through the aorta, and to a location in the aorta proximate the heart of the patient.

10. The system of claim 1, further comprising:
a retrieval sheath configured to access the elongate shaft extending through the axillary artery and move the elongate shaft into the aorta, and
wherein the retrieval sheath is configured to extend over the blood pump during removal of the blood pump from the patient through a femoral artery access site.

11. A method comprising:
inserting a pump to a heart of a patient through a femoral artery access site using a delivery sheath;
removing the delivery sheath from an elongate shaft extending from the pump while leaving the pump in place;
accessing the elongate shaft of the pump via an axillary artery of the patient; and
coupling the elongate shaft with a controller.

12. The method of claim 11, further comprising:
positioning the elongate shaft through an axillary artery access site.

13. The method of claim 11, wherein accessing the elongate shaft of the pump via the axillary artery comprises snaring the elongate shaft while the elongate shaft is extending along an aorta of the patient.

14. The method of claim 13, further comprising:
pulling the elongate shaft through the axillary artery.

15. The method of claim 11, further comprising:
initiating operation of the pump to treat a cardiogenic shock condition of the patient while allowing patient to be ambulatory.

16. The method of claim 11, further comprising:
detaching the elongate shaft from the pump and removing the elongate shaft from vasculature of the patient through the axillary artery.

17. The method of claim 11, further comprising:
engaging the elongate shaft with a retrieval sheath extending through a femoral artery access site while the elongate shaft is extending along the axillary artery.

18. A method comprising:
operating a large-bore blood pump extending into a heart of a patient via an elongate shaft coupled with the large-bore blood pump and extending through an axillary artery of the patient; and
removing the large-bore blood pump from the patient via a femoral artery access site.

19. The method of claim 18, further comprising:
grasping the elongate shaft extending through the axillary artery of the patient, and
wherein the elongate shaft is removed from the patient via the femoral artery access site with the large-bore blood pump.

20. The method of claim 18, further comprising:
detaching the elongate shaft from the large-bore blood pump; and
removing the elongate shaft from vasculature of the patient via an axillary artery access site.
